(19) United States
(12) Patent Application Publication
(10) Pub. No.: US 2024/0409483 A1
(43) Pub. Date: Dec. 12, 2024

(54) Integrated Energy Systems For The Direct Capture Of Carbon Dioxide From Emissions Sources For Methanol Production

(71) Applicant: NuScale Power, LLC, Corvallis, OR (US)

(72) Inventors: Francis Y. Tsang, Bellingham, WA (US); José N. Reyes, JR., Corvallis, OR (US); Luis DePavia, Corvallis, OR (US)

(21) Appl. No.: 18/737,623

(22) Filed: Jun. 7, 2024

Related U.S. Application Data
(60) Provisional application No. 63/507,057, filed on Jun. 8, 2023.

(51) Int. Cl. C07C 29/151; C02F 1/44; C02F 103/08; C25B 1/02; C25B 1/23

(52) U.S. Cl. CPC C07C 29/1518; C02F 1/441; C25B 1/02; C25B 1/23; C02F 2103/08

(57) ABSTRACT

Described herein are techniques that may be performed in an Integrated Energy System (IES). The IES may include a power plant, an emission source, and a chemical processing plant. The IES may include one or more sub-plants configured to receive Carbon Dioxide from an emission source, convert a first portion of the Carbon Dioxide into Carbon Monoxide, receive Sodium Hydroxide, combine the Carbon Monoxide and the Sodium Hydroxide to produce Hydrogen gas, and combine, using a reaction chamber, a second portion of the Carbon Dioxide, the Carbon Monoxide, and the Hydrogen to produce Methanol.

### CROSS-REFERENCE TO RELATED APPLICATIONS

This application claims the benefit of U.S. Provisional Patent Application No. 63/507,057, filed Jun. 8, 2023, and titled “NUCLEAR REACTOR INTEGRATED ENERGY SYSTEMS FOR THE DIRECT CAPTURE OF CARBON DIOXIDE FROM EMISSIONS SOURCES FOR METHANOL PRODUCTION” which is incorporated herein by reference in its entirety.

### TECHNICAL FIELD

The present technology is directed to nuclear reactor integrated energy systems (IESs) for energy production and green industrial applications, such as capturing Carbon Dioxide and producing green Methanol and associated devices and methods.

### BACKGROUND

Cumulative Carbon Dioxide (CO₂) emissions are the dominant driver of climate change. Most CO₂ emissions are produced from burning fossil fuels for electricity, for use to produce thermal and steam, and for transportation. The seven largest CO₂ emission industries in the world are: (1) power plants (coal, natural gas, oil fired), (2) oil refinery plants, (3) ammonia production plants, (4) chemical manufacturing and production plants, (5) cement production plants, (6) steel manufacturing plants, and (7) transportation. President Biden has set a goal for the United States to fully produce carbon-free electricity by 2035. The proposed new EPA rules are expected to help speed up the progress of carbon capture and give utilities options for cutting Carbon Dioxide (CO₂) emissions, such as by installing new carbon-capture systems or switching to cleaner fuels. Despite the new rules, tax credits, and trends towards increased deployment of renewable energy however, electric utilities have been reluctant to adopt new technologies and methods because of their high cost. Therefore, fossil fuels will continue to play a significant role in global energy supply and in industrial processes for many years to come.

There is a need to develop methods of integrating carbon capture into present industrial operations to address the challenges of climate change. Carbon sequestration from atmospheric Carbon Dioxide (CO₂) and industrial emissions pose significant challenges, however. For example, Direct Air Capture (DAC) and Carbon Capture, Use and Storage (CCUS) facilities are capital-intensive to deploy and energy-intensive to operate. In both scenarios, the energy used to capture the Carbon Dioxide (CO₂) will determine if and how net-negative the system is for carbon and can also be a significant determinant of the cost per tonne of Carbon Dioxide (CO₂) captured. The choice of location also needs to be based on the energy source needed to run a DAC or CCUS plant. Success of these technologies from an economic and regulatory perspective, therefore, relies on the availability of local low-carbon energy sources. Therefore, there is a need to address the challenges of climate change by developing methods for reducing or eliminating Carbon Dioxide (CO₂) emissions in power production and by integrating carbon capture technologies with industrial processes.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 is a schematic diagram of an integrated energy system for the production of clean water and capture of Carbon Dioxide (CO₂), in accordance with at least some embodiments.

FIG. 2 depicts a schematic diagram of an integrated energy system for the capture and conversion of Carbon Dioxide (CO₂) from an emission source, in accordance with at least some embodiments.

FIG. 3 is a block diagram illustrating the utilization of a Solid Oxide Electrolysis Cell (SOEC), in accordance with at least some embodiments.

FIG. 4 depicts a block diagram illustrating a chlor-alkali membrane process in accordance with at least some embodiments.

FIG. 5 depicts a block diagram illustrating a Hydrogen (H₂) and Sodium Oxalate (Na₂C₂O₄) Plant, in accordance with at least some embodiments.

FIG. 6 is a block diagram illustrating an example Methanol production plant, in accordance with at least some embodiments.

FIG. 7 is a schematic view of a nuclear power plant system including multiple nuclear reactors in accordance with embodiments of the present technology.

FIG. 8 is a partially schematic, partially cross-sectional view of a nuclear reactor system configured in accordance with embodiments of the present technology.

FIG. 9 is a partially schematic, partially cross-sectional view of a nuclear reactor system configured in accordance with additional embodiments of the present technology.

FIG. 10 illustrates an example process for capturing carbon and the production of Methanol (CH₃OH), in accordance with additional embodiments of the present technology.

### DETAILED DESCRIPTION

The present technology provides devices and methods for removing Carbon Dioxide (CO₂) directly from an emissions source, such as from natural-gas or coal-fired power generation plants, chemical product production plants, oil refineries, ore processing plants, steel processing plants, and/or other generators of green-house gases. In embodiments, the present disclosure is directed to techniques that may be performed in relation to Integrated Energy Systems (IESs), such as for use in green industrial processes that produce few or no carbon emissions, to capture carbon from an emission source, for resource production, and associated devices and methods. IESs of the present technology may include a power plant (e.g., a primary power plant) that is integrated with one or more industrial processes and resource production plants to provide power with few or no carbon emissions, to capture Carbon Dioxide (CO₂) directly from an emissions source, and to produce resources from the captured Carbon Dioxide (CO₂).

Industrial processes in accordance with embodiments of the present technology may include water purification, chemical manufacturing and production, natural-gas or coal-fired power generation plants, petroleum and oil refining, bulk plastic waste recycling and gasification, cement production, ore processing plants, steel and primary metal manufacturing, transportation, food processing, pharmaceutical production, pulp and paper, materials manufacturing, and/or other industrial plants. Such an IES may be capable of providing electricity and steam, or a combination of both, from the power plant to the industrial processes for carbon capture and resource production, such as the production of chemical products. The IES of the present disclosure can also assist industries to meet EPA and other national and global regulations for cutting Carbon Dioxide (CO₂) emissions. In embodiments, the IES may be modular and therefore may be retrofit to existing industrial processes for carbon capture and resource production.

Because of the drive toward cleaner and more efficient forms of power production, nuclear power will be increasingly important in the coming years. In operation, nuclear power plants use the Nuclear fission process to generate heat, which is then used to produce steam to turn turbines and generate electricity. This process can result in the production of electrical power that reduces the need for coal and natural gas to produce electricity. Nuclear power plants provide reliable baseload power without emitting greenhouse gases such as Carbon Dioxide (CO₂) during operation, making them attractive for countries that are seeking to reduce carbon emissions and enhance energy security. Due to the advantages of nuclear energy for providing electricity, the present disclosure presents novel methods of using nuclear power in integrated energy systems for carbon capture and “green” resource production, such as the production of “green” chemical products.

In some embodiments, an IES includes a power plant system having multiple small modular nuclear reactors (SMRs) specifically configured to operate in unison to support one or more of the industrial processes. SMRs are nuclear reactors that are smaller in terms of size (e.g., dimensions) and power compared to large, conventional nuclear reactors. Moreover, they are modular in that some or all of their systems and components can be factory-assembled and transported as a unit to a location for installation. In some aspects of the present technology, the multiple SMRs of the integrated energy system can flexibly and dynamically provide electricity, steam, or a combination of both electricity and steam to the industrial processes due to the modularity and flexibility of the SMRs. That is, a configuration of the SMRs can be switched during operation to provide varying levels of steam and electricity output depending on the operational states and/or demands of the industrial processes.

In embodiments, a power plant of the present disclosure can be a permanent or temporary installation built at or near (e.g., roughly 1 km from) the location of an industrial process facility or can be a mobile or partially mobile system that is moved to and assembled at or near the location of the industrial process facility. More generally, the power plant can be local (e.g., positioned at or near) to the industrial processes/operations it supports. For example, the power plant can be located within 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant is configured to supply a portion of electricity to a power grid.

In some embodiments, the present disclosure includes systems and methods that may address many problems associated with conventional Carbon Dioxide (CO₂) capture and storage solutions, such as economic viability. In embodiments, the IES of the present disclosure may capture Carbon Dioxide (CO₂) from an industrial emission source and utilize the captured Carbon Dioxide (CO₂) in the production of one or more resources and/or products. In embodiments, resources and/or products produced in the IES may be sold directly to industry. In embodiments, resources and/or products within the IES may be further utilized in the production of other resources and/or products that can be used within the IES or sold to industry. The utilization of captured Carbon Dioxide (CO₂) to produce saleable resources and/or products thereby decreases the cost per tonne of Carbon Dioxide (CO₂) captured.

In some embodiments, the IESs may capture Carbon Dioxide (CO₂) from an emissions source and process the captured Carbon Dioxide (CO₂) in one or more resource production plants to produce saleable resources and/or products. For example, the IES may be operably coupled to a Methanol production plant wherein the captured Carbon Dioxide (CO₂) is converted to Methanol (CH₃OH). Methanol (CH₃OH), also known as methyl alcohol, is a highly versatile chemical widely used for industrial purposes and prevalent in our everyday lives. It is a base material in the production of acetic acid and formaldehyde, and also increasingly being used in ethylene and propylene production. Methanol (CH₃OH) is one of the most prolific intermediate materials for the production of other chemicals and materials. In the chemical industry Methanol (CH₃OH) mainly serves as a raw material in the production of formaldehyde, olefins, acetic acid, MTBE, DME, as well as biodiesel. So, renewable Methanol (CH₃OH) is a pre-requisite for making a broad range of chemical products green such as polymer fibers for the textile industry, plastics for packaging, glues, adsorbents/diapers, paints, adhesives, solvents, and much more. Besides its use in the chemical, construction and plastics industries, Methanol (CH₃OH) also serves as a fuel or fuel additive.

The conventional production method for Methanol (CH₃OH) involves a catalytic process using fossil feedstock such as natural gas, coal, or syngas. Currently, most Methanol (CH₃OH) is produced from the catalytic conversion of Carbon Dioxide (CO₂) and Hydrogen (H₂). The predominant method of producing Carbon Dioxide (CO₂) and Hydrogen (H₂) for Methanol (CH₃OH) production is through the steam-methane reforming of natural gas. The steam-reforming process, however, results in significant Carbon Dioxide (CO₂) emissions into the atmosphere. The catalytic hydrogenation of Carbon Dioxide (CO₂) for the production of Methanol (CH₃OH) also results in water formation as a byproduct, which leads to kinetic inhibition and accelerated deactivation of the Cu/ZnO catalysts. To counteract the formation of water and prevent the deactivation of the Cu/ZnO catalysts, Carbon Monoxide (CO) is required in the reaction chamber to remove water via a Water-Gas-Shift (WGS) reaction.

In embodiments of the present disclosure, the IES can produce Carbon Dioxide (CO₂), Hydrogen (H₂), and Carbon Monoxide (CO) for the production of Methanol (CH₃OH). In embodiments, Carbon Dioxide (CO₂) may be captured from an emission source and separated from oxides, particulates, and other contaminants in a separator, such as a scrubber, Carbon Monoxide (CO) gas for the Water-Gas-Shift (WGS) reaction may be generated from the captured Carbon Dioxide (CO₂) in a Solid Oxide Electrolysis Cell (SOEC), and Hydrogen (H₂) may be formed from the byproducts of industrial processes.

In some embodiments, the IES is operably coupled to a water source, such as an ocean. In embodiments, water from the water source is fed to a water purification plant, such as a Reverse Osmosis (RO) desalination plant, configured to produce clean water. As climate change progresses, water scarcity will become an increasing threat to nations and individuals throughout the world. Nearly 30% of the human population live in water-stressed countries, and 9% live in critically water-stressed countries. Desalination has been successfully implemented in many regions mitigating the negative effects of water scarcity by providing clean high-quality water for the local populations. Desalination, however, is a resource and energy intensive process with the most commonly used desalination process, Reverse Osmosis (RO), requiring between 3.44-22.36 kWh/m³ of freshwater produced. Globally, RO accounts for around 69% of installed desalination capacity or about 69 million m³/day, thus this equates to approximate emissions of 33 billion kg of Carbon Dioxide (CO₂) per year for clean RO water. Therefore, there is a need to develop integrated energy systems that produce few, or no carbon emissions, to reduce the carbon burden of clean water production.

Desalination of seawater also produces large quantities of brine as a by-product. Brine is a high concentration salt and water solution, primarily Sodium Chloride (NaCl) ranging from about 5% to about 26%. Brine is denser than seawater and therefore sinks to the bottom of the ocean and if released directly, can damage ecosystems. The desalination of seawater through RO on average produces about 1.4 liters of brine for every liter of clean water. Or in other words, for RO to produce the 69 million m³/day of fresh water on a global scale, there must be 97 million m³/day of brine that requires proper environmental disposal. Therefore, there is a need to develop integrated energy systems that produce few, or no carbon emissions, that can address the current burden of brine formation in the desalination of sea water.

In embodiments, the present disclosure includes methods and devices that may address many problems associated with conventional clean water production solutions such as the production of clean water with few or no carbon emissions. In embodiments, the IES of the present disclosure may utilize byproducts of clean water production, such as brine from an RO desalination plant, in resource production. In embodiments, the IES may convert brine converted into useful and/or saleable products and saline water with low salt concentration that may be re-cycled to a water purification facility, such as a desalination facility. In embodiments, resources and/or byproducts produced in the IES may be sold directly to industry. In embodiments, byproducts and/or resources may be further processed to produce other resources that can be used within the IES or sold to industry, thereby decreasing the cost per tonne of clean water produced.

Aspects of the present disclosure include treating brine from a water purification plant, such as a Reverse Osmosis (RO) desalination plant, and converting it to useful industrial chemicals. In some embodiments, brine is fed to a chlor-alkali membrane process. The chlor-alkali process is an electrolysis process that has been demonstrated to treat brine and to produce Sodium Hydroxide (NaOH) solution, Chlorine (Cl₂) gas and Hydrogen (H₂) gas from the Sodium Chloride (NaCl) brine solution and clean water. The clean water used in the chlor-alkali process can be supplied directly from the RO plant. The produced Chlorine (Cl₂) gas and Hydrogen (H₂) gas may be removed as a product or used in further resource production, such as combining the Chlorine (Cl₂) gas and Hydrogen (H₂) gas to form Hydrogen Chloride (HCl) gas which can be converted to Hydrochloric Acid (HCl). The produced Sodium Hydroxide (NaOH) solution may be removed as a product or used in further resource production, such as the production of Hydrogen (H₂).

In embodiments, Sodium Hydroxide (NaOH) solution from the chlor-alkali membrane process may be fed to an oven, such as a Thermal Vacuum Dehydration Chamber, to form solid Sodium Hydroxide (NaOH). The solid Sodium Hydroxide (NaOH) may then be fed to a reaction chamber and introduced to Carbon Monoxide (CO) under pressure, for example from the Solid Oxide Electrolysis Cell (SOEC), where the reaction produces Sodium Formate (HCOONa). The Sodium Formate (HCOONa) may then undergo thermal decomposition to produce Hydrogen (H₂) and Sodium Oxalate (Na₂C₂O₄). The Sodium Oxalate (Na₂C₂O₄) may be sold to industry or used in further resource production, for example as a component for water treatment, food additives, and the production of other chemicals. The Hydrogen (H₂) may be fed to a reaction chamber with Carbon Dioxide (CO₂) and Carbon Monoxide (CO), for example from the SOEC, for the formation of Methanol (CH₃OH). Byproducts from the SOEC, such as oxygen, may be sold to industry sold to industry or used in further resource production.

The current industrial process for the production of Methanol (CH₃OH) is the steam-methane reforming process. This process is energy intensive and produces a significant amount of Carbon Dioxide (CO₂) emissions. The integration of the above steps for the capture and utilization of industrial Carbon Dioxide (CO₂) for the production of Methanol (CH₃OH) and other saleable resources using carbon free power provides enumerable benefits to the resent state of the art. In embodiments, an IES of the present disclosure receives as input, electricity, saline water, and Carbon Dioxide (CO₂) from an emissions source, and converts these inputs into useful and saleable products such as clean water, Methanol (CH₃OH), Hydrogen Chloride (HCl) gas and Hydrochloric Acid (HCl) via the Hydrogen (H₂) and Chlorine (Cl₂) gasses, Sodium Oxalate (Na₂C₂O₄), Oxygen (O₂), Sodium Hydroxide (NaOH), Chlorine (Cl₂), Hydrogen (H₂), and Sodium Formate (HCOONa). In embodiments, the IES produces minimal emissions, waste, and byproducts. In embodiments, the IES generates no carbon emissions and results in net-negative carbon emissions through the capture and use of Carbon Dioxide (CO₂) from an emissions source.

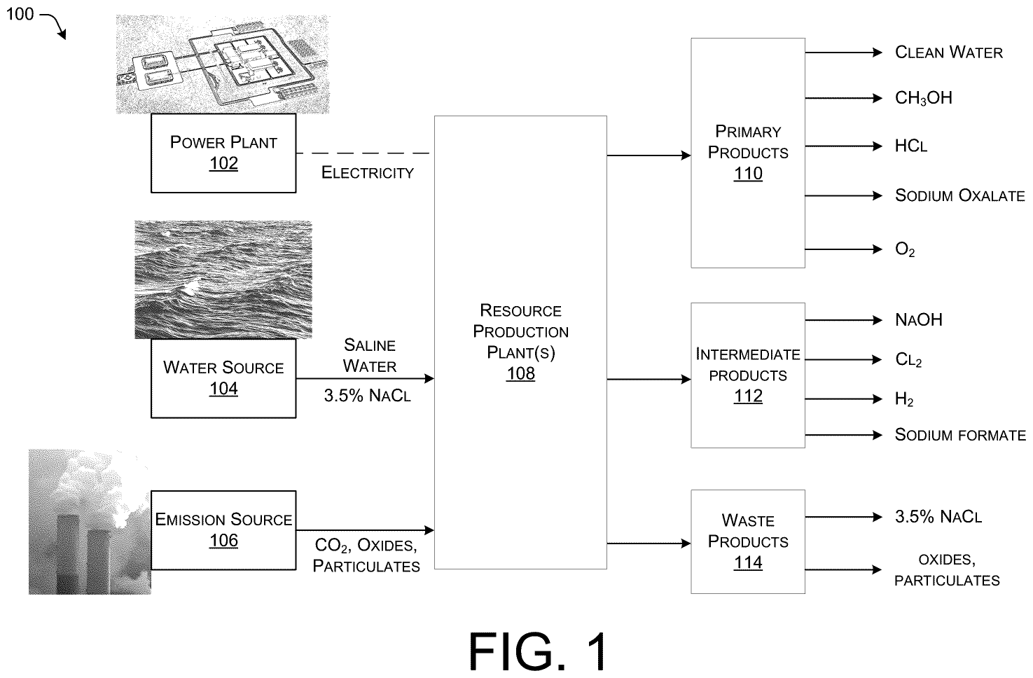

Certain details are set forth in the following description and in FIGS. 1-10 to provide a thorough understanding of various embodiments of the present technology. In other instances, well-known structures, materials, operations, and/or systems often associated with nuclear reactors, power plant systems, integrated energy systems, chemical production plants, industrial process plants, electrolysis systems, Hydrogen and Oxygen production plants, direct air capture (DAC) plants, oil refineries, and the like, are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. The terminology used below is to be interpreted in its broadest reasonable manner, even though it is being used in conjunction with a detailed description of certain examples of embodiments of the technology.

The accompanying Figures depict embodiments of the present technology and are not intended to limit its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

Each of the references cited herein is incorporated herein by reference in its entirety. However, to the extent any materials incorporated herein by reference conflict with the present disclosure, the present disclosure controls. The headings provided herein are for convenience only and should not be construed as limiting the subject matter disclosed.

FIG. 1 is a schematic diagram of an Integrated Energy System (IES) 100 of the present disclosure. In embodiments, the IES 100 receives as input electricity from a power plant 102, saline water from a water source 104, and Carbon Dioxide (CO₂) from an emission source 106. In embodiments, the IES 100 includes one or more resource production plants 108 that are configured to convert the inputs into useful and saleable products.

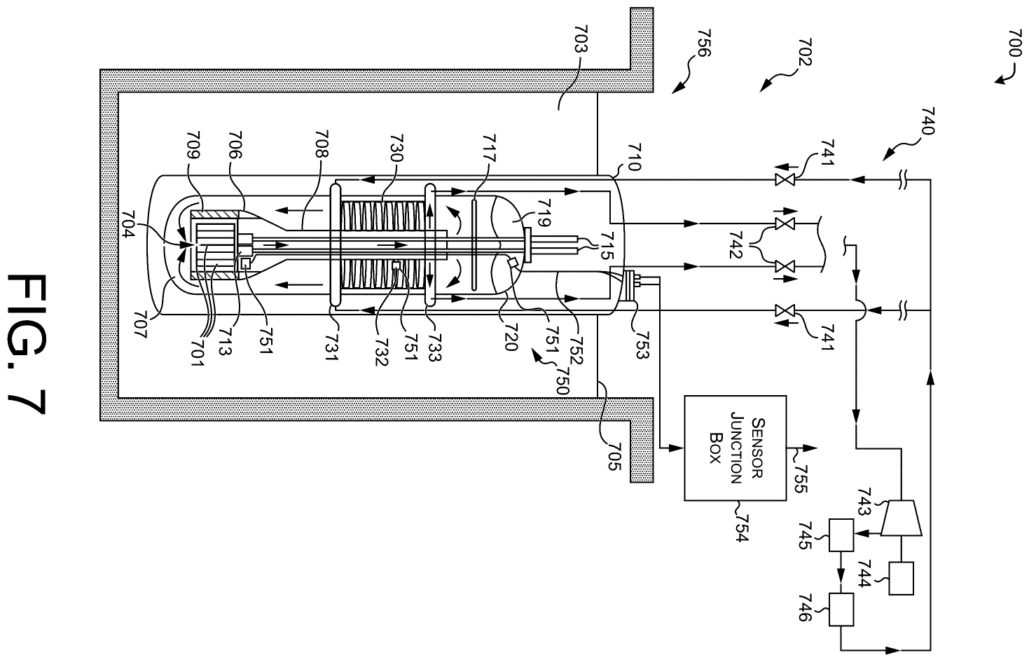

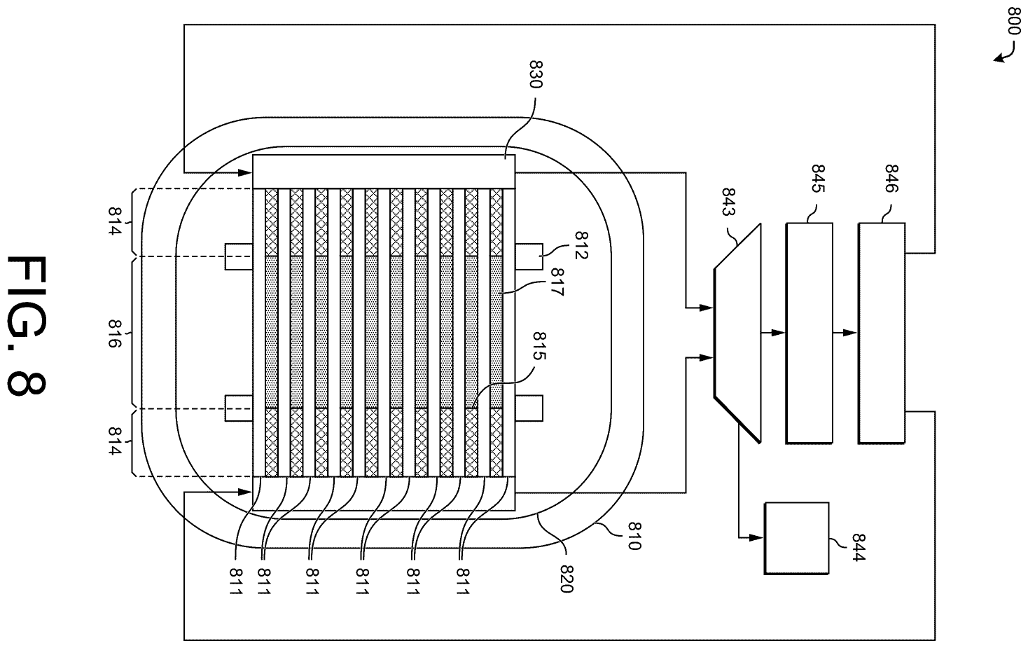

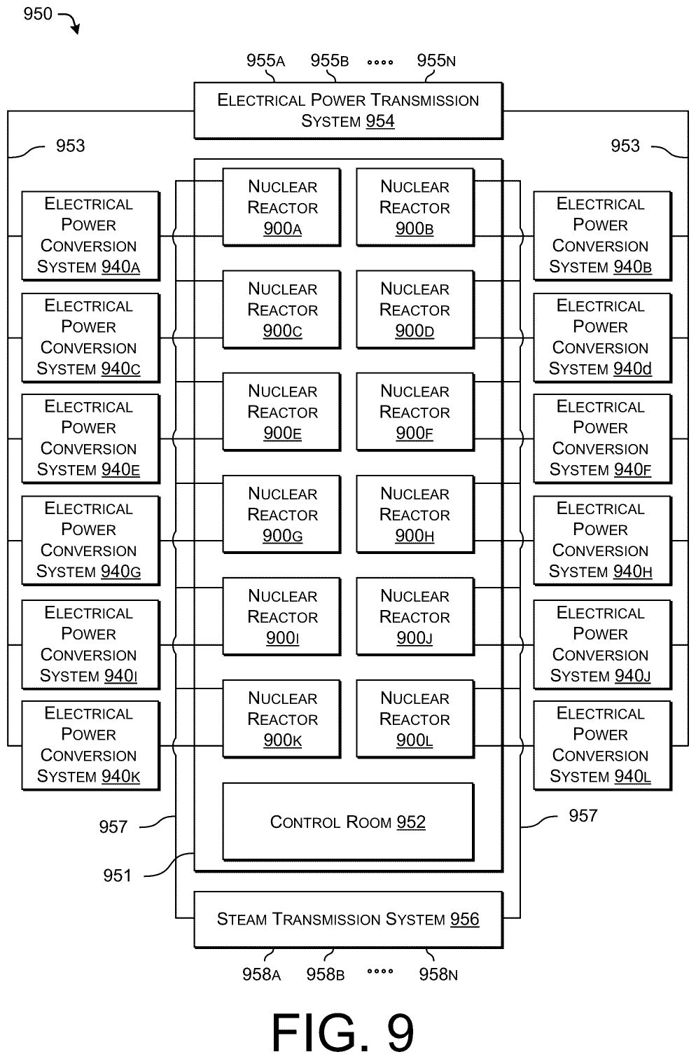

In embodiments, the IES 100 is operably coupled to the power plant 102 and the power plant 102 is configured to supply power to the IES 100. In embodiments the power plant 102 may include the power plant power plant system 750 of FIG. 7, in accordance with additional embodiments of the present technology. The power plant 102 may include a nuclear power module (NPM) including one or more light water nuclear reactor (LWR), one or more small modular nuclear reactor (SMR), and any reactor 700 of FIG. 7, reactor system 800 of FIG. 8, and reactor system 900 of FIG. 9.

In embodiments, the IES 100 is operably coupled to the water source 104. In embodiments, the water source 104 includes a natural body of water, such as an ocean, a sea, or a lake, a storage tank, an industrial process, and a water treatment facility. In embodiments, the water source 104 provides saline water, such as sea water, brackish water, and brine, to the IES 100. In embodiments, the saline water may have a dissolved salt concentration of 0.05% to 50%. For example, sea water from the world's oceans has a salt concentration of about 3.5%, and saltwater lakes and seas around the world have salt concentrations from 0.59% to 50%. In embodiments, the saline water includes salts such as Sodium Chloride (NaCl).

In embodiments, the IES 100 is operably coupled to the emission source 106 and configured to capture carbon in the form of Carbon Dioxide (CO₂) from the emission source 106. In embodiments, the emission source 106 can include natural-gas or coal-fired power generation plants, chemical product production plants, oil refineries, ore processing plants, steel processing plants, and/or other industrial plants.

In embodiments, the IES 100 produces primary products 110 in the one or more resource production plants 108. In embodiments, primary products 110 may include clean water, Methanol (CH₃OH), Hydrogen Chloride (HCl) gas, Hydrochloric Acid (HCl), Sodium Oxalate (Na₂C₂O₄), and Oxygen (O₂). In some cases, the primary products 110 may be stored and/or sold to consumers. In embodiments, a portion of any one of the primary products 110 may be recycled within the IES 100. In embodiments, the IES 100 produces intermediate products 112 in the one or more resource production plants 108. In embodiments, intermediate products 112 may include including Sodium Hydroxide (NaOH) solution, Sodium Hydroxide (NaOH) solid, Chlorine (Cl₂), Hydrogen (H₂), and Sodium Formate (HCOONa). In embodiments, the intermediate products 112 may be recycled within the IES 100. In some cases, a portion of any one of the intermediate products 112 may be stored and/or sold to consumers. In embodiments, the IES 100 may produce waste products 114 including low concentration salt water (e.g., 3.5% Sodium Chloride (NaCl)) and contaminants separated from the Carbon Dioxide (CO₂) from the emission source 106, such as particulates, sulfur oxides, and other gases. In embodiments, the waste products 114 may be recycled within the IES 100. In some cases, all, or a portion of any one of the waste products 114 may be stored, sold to consumers, further treated for safe disposal, and/or safely released into the environment. In embodiments, the IES 100 produces minimal emissions, waste, and byproducts. In embodiments, the IES 100 generates no carbon emissions and results in net-negative carbon emissions through the capture and use of Carbon Dioxide (CO₂) from the emission source 106.

In embodiments, the power plant 102 can be a permanent or temporary installation built at or near (e.g., roughly 1 km from) the location of the water source 104, emissions source 106, and/or resource production plants 108 or can be a mobile or partially mobile system that is moved to and assembled at or near the location of the water source 104, emissions source 106, and/or resource production plants 108. More generally, the power plant 102 can be local (e.g., positioned at or near) to the industrial processes/operations it supports. For example, the power plant can be located within 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant 102 is configured to supply a portion of electricity to a power grid.

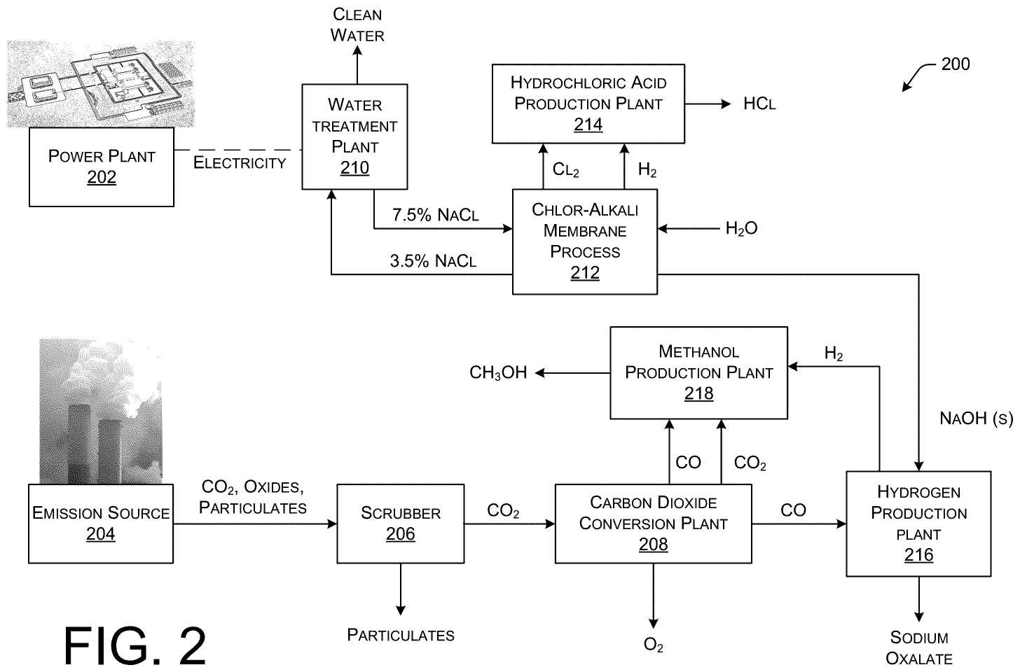

FIG. 2 is a schematic diagram of an Integrated Energy System (IES) 200 of the present disclosure. In embodiments, the IES 200 is operably coupled to a power plant 202. In embodiments, the power plant 202 is configured to supply power to the IES 200. In embodiments the power plant 202 may include the power plant power plant system 750 of FIG. 7, in accordance with additional embodiments of the present technology. The power plant 202 may include a nuclear power module (NPM) including one or more light water nuclear reactor (LWR), one or more small modular nuclear reactor (SMR), and any reactor 700 of FIG. 7, reactor system 800 of FIG. 8, and reactor system 900 of FIG. 9.

In some embodiments, the IES 200 is operably coupled to one or more emission sources 204 and configured to capture carbon in the form of Carbon Dioxide (CO₂) from the emission source 204 for use in resource production. In embodiments, the emission source 204 may include natural-gas or coal-fired power generation plants, chemical product production plants, oil refineries, ore processing plants, steel processing plants, and/or other industrial plants.

In embodiments, Carbon Dioxide (CO₂) emissions from the emission source 204 may be fed to one or more scrubbers 206. The one or more scrubbers 206 may be configured to separate Carbon Dioxide (CO₂) from particulates, sulfur oxides, other gases, and contaminants emitted by the emission source 204. The one or more scrubbers 206 may include a wet or dry scrubber to remove oxides of sulfur. The one or more scrubbers 206 may include a filter to remove particulates, and/or a fly ash remover. In embodiments, the scrubber 206 is configured to receive power from the power plant 202.

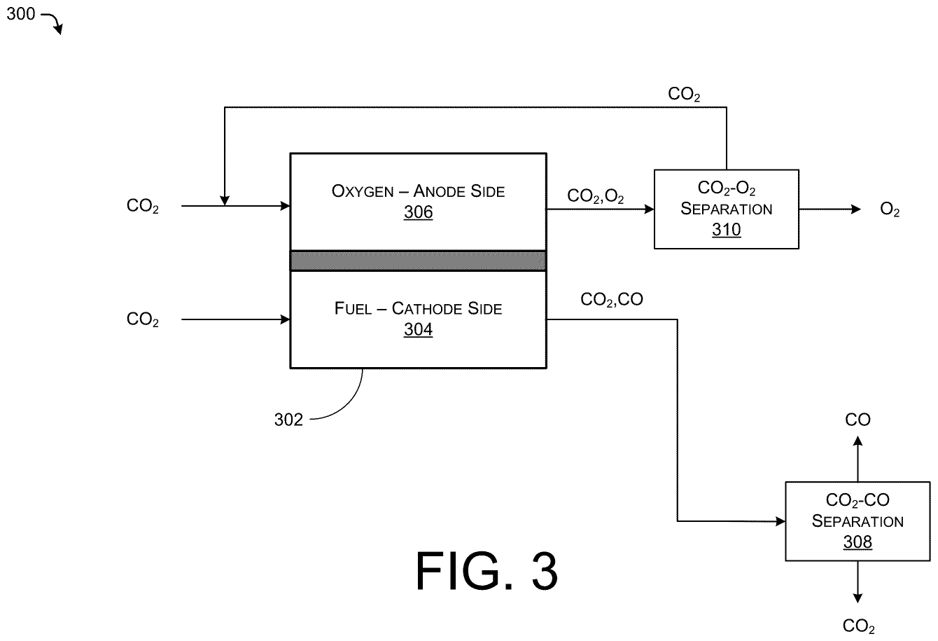

In embodiments, the IES 200 may include a Carbon Dioxide (CO₂) conversion plant 208 configured to convert Carbon Dioxide (CO₂) into Carbon Monoxide (CO) and Oxygen (O₂). In embodiments, the Carbon Dioxide (CO₂) conversion plant 208 may include the Carbon Dioxide (CO₂) conversion plant 300 of FIG. 3. In embodiments, Carbon Dioxide (CO₂) from the scrubber 206 may be fed to the Carbon Dioxide (CO₂) conversion plant 208. In embodiments, the Carbon Dioxide (CO₂) conversion plant 208 may include a one or more Solid Oxide Electrolysis Cells (SOEC) to convert Carbon Dioxide (CO₂) into Carbon Monoxide (CO) and Oxygen (O₂). In embodiments, the Carbon Dioxide (CO₂) conversion plant 208 may include one or more additional separation processes, such as Pressure Swing Adsorption (PSA), membrane, and/or cryogen separation, for separating Carbon Dioxide (CO₂) from Carbon Monoxide (CO). In embodiments, the Carbon Dioxide (CO₂) conversion plant 208 may include one or more separation processes, such as pressure swing absorption (PSA), membrane, and/or cryogen separation, for separating Carbon Dioxide (CO₂) from Oxygen (O₂). In embodiments, Carbon Dioxide (CO₂) within the Carbon Dioxide (CO₂) conversion plant 208 may be recycled to increase conversion efficiency. Oxygen (O₂) produced in the Carbon Dioxide (CO₂) conversion plant 208 may be stored, further purified, used in downstream processes, and/or sold. Carbon Dioxide (CO₂) and Carbon Monoxide (CO) from the Carbon Dioxide (CO₂) conversion plant 208 may be fed to downstream processes or recycled within the IES 200 for resource production. In embodiments, the Carbon Dioxide (CO₂) conversion plant 208 is configured to receive power and/or thermal energy from the power plant 202.

In embodiments, the IES 200 includes a water treatment plant 210. The water treatment plant 210 may include one or more water treatment processes, such as Reverse Osmosis (RO), distillation, and filtration. The water treatment plant 210 may receive water from a water source. In embodiments, the water source includes a natural body of water, such as an ocean, a sea, or a lake, a storage tank, an industrial process, and a separate water treatment facility. In embodiments, the water source provides saline water, such as sea water, brackish water, and brine, to the water treatment plant 210. In embodiments, the saline water may have a dissolved salt concentration of 0.05% to 50%. For example, sea water from the world's oceans has a salt concentration of about 3.5%, and saltwater lakes and seas around the world have salt concentrations from 0.59% to 50%. In embodiments, the saline water includes salts such as Sodium Chloride (NaCl). In embodiments, the water treatment plant 210 is configured to receive power from the power plant 202.

In embodiments, the water treatment plant 210 produces clean water and brine. In the illustrated example, saline water, such as sea water with a salt concentration of 3.5% Sodium Chloride (NaCl), is fed to the water treatment plant 210. The water treatment plant 210 converts the saline water into clean water (e.g., <0.05% Sodium Chloride (NaCl) concentration) and brine (e.g., 7.5% Sodium Chloride (NaCl) concentration).

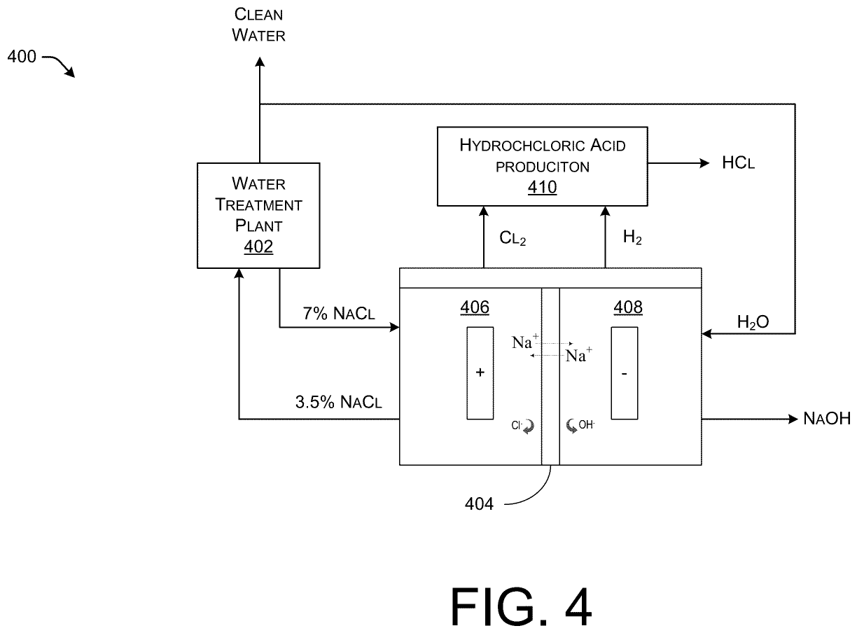

In embodiments, brine (e.g., 7.5% Sodium Chloride (NaCl) concentration) from the water treatment plant 210 is fed to a chlor-alkali membrane process 212 such as the chlor-alkali membrane process 400 in FIG. 4. In embodiments, a portion of the clean water from the water treatment plant 210 may be fed to chlor-alkali membrane process 212.

In embodiments, the chlor-alkali membrane process 212 may include an ion-selective membrane configured to allow Sodium ions (Na+) to flow freely across the membrane, while Chloride ions (Cl⁻) and Hydroxide ions (OH⁻) are prevented from migrating across the membrane. The chlor-alkali membrane process 212 may include an anode and a cathode. At the anode, Chloride ions (Cl⁻) from the brine solution are oxidized to form Chlorine (Cl₂) gas. At the cathode, water (H₂O) is reduced to Hydroxide ions (OH⁻) and Hydrogen (H₂) gas, releasing Hydroxide ions (OH⁻) into the solution. Sodium ions (Na⁺) from the brine solution flow across the membrane toward the cathode and combine with Hydroxide ions (OH⁻) to produce a Sodium Hydroxide (NaOH) solution. The Sodium Hydroxide (NaOH) solution may be removed as a product from the chlor-alkali membrane process 212.

In embodiments, the chlor-alkali membrane process 212 can reduce the Sodium Chloride (NaCl) concentration of brine. The outlet stream from the chlor-alkali membrane process 212 may, for example, be reduced to a benign sea water concentrations of Sodium Chloride (NaCl) (e.g., 3.5%) and may be further processed in a downstream chlor-alkali membrane cell, or fed back to the water treatment plant 210.

In embodiments, the Chlorine (Cl₂) gas and Hydrogen (H₂) gas generated in the chlor-alkali membrane process 212 may be removed as a product to be stored, sold, or used in further resource production, such as in a Hydrochloric Acid production plant 214.

In embodiments, the water treatment plant 210, the chlor-alkali membrane process 212, and the Hydrochloric acid production plant 214 are configured to receive power from the power plant 202.

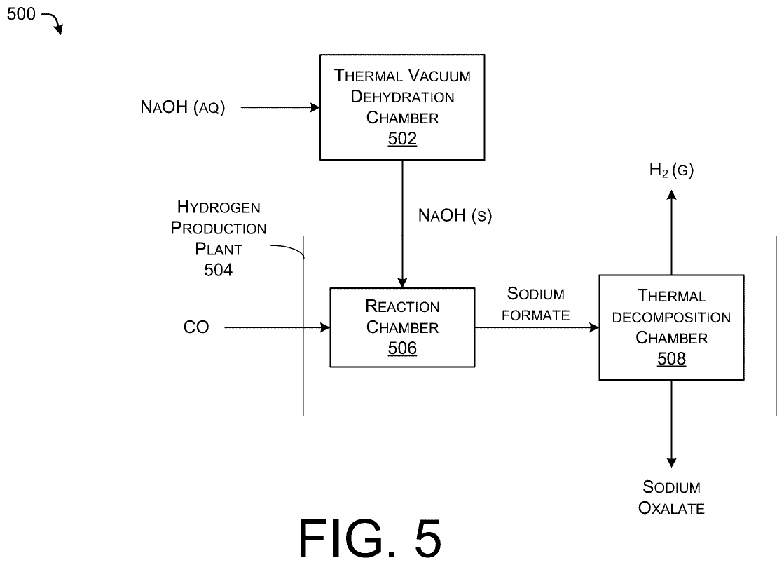

In embodiments, the IES 200 may include a Hydrogen production plant 216, such as the Hydrogen production process 500 in FIG. 5, in accordance with embodiments of the present disclosure. In embodiments, Carbon Monoxide (CO) from the Carbon Dioxide conversion plant 208 is fed to the Hydrogen production plant 216, along with solid Sodium Hydroxide (NaOH). In embodiments, Sodium Hydroxide (NaOH) solution, for example from the chlor-alkali membrane process 212, may be converted to a solid form through a drying process, such as a Thermal Vacuum Dehydration Chamber. In the Hydrogen production plant 216, Carbon Monoxide (CO) is absorbed by the solid Sodium Hydroxide (NaOH) under pressure to produce Sodium Formate (HCOONa), for example at a temperature of 130° C. and a pressure between 6-8 bar. In embodiments, the Sodium Formate (HCOONa) may then be heated to undergo thermal decomposition and thereby produce Hydrogen (H₂) gas and Sodium Oxalate (Na₂C₂O₄). The temperature of Sodium Formate (HCOONa) thermal decomposition starting at about 330° C.

Hydrogen (H₂) generated in the Hydrogen production plant 216 may be sent to downstream processes for use in resource production, may be stored and/or sold. Sodium Oxalate (Na₂C₂O₄), as well as any intermediate or byproducts, generated in the Hydrogen production plant 216 may be recycled within the IES 200 to optimize yield, may be sent to downstream processes for use in resource production, and may be stored and/or sold. In embodiments, the Hydrogen production plant 216 is configured to receive power from the power plant 202.

In embodiments, the IES 200 may include a Methanol production plant 218, such as the Methanol production process 600, in accordance with embodiments of the present disclosure. The Methanol production pant 218 may include a high selectivity Copper (Cu) and zinc oxide (ZnO) based catalyst (Cu/ZnO). The reaction of Carbon Dioxide (CO₂) and Hydrogen (H₂) generate water which accelerates the deactivation of the Cu/ZnO catalyst. Introduce Carbon Monoxide (CO) into the reaction of Carbon Dioxide (CO₂) and Hydrogen (H₂) removes water via the Water-Gas-Shift (WGS) reaction. In embodiments, Carbon Dioxide (CO₂), Carbon Monoxide (CO) and Hydrogen (H₂) are fed into the Methanol production plant 218. Carbon Dioxide (CO₂) and Carbon Monoxide (CO) may, for example, be fed from the Carbon Dioxide (CO₂) conversion plant 208. Hydrogen (H₂), for example, may be fed from the Hydrogen production plant 216. Methanol (CH₃OH) may be stored or sold. In embodiments, the Methanol production plant 216 is configured to receive power from the power plant 202.

In embodiments, the IES 200 may include a control system. In representative embodiments, the IES 200 has a steady source of electric power from the power plant 202 and saline water, available all the time at the desired quantity. The concentration and presence of Carbon Dioxide (CO₂) may vary throughout the day depending on the operating conditions of the emission source 204. The optimal production rate of Sodium Formate (HCOONa) and Methanol (CH₃OH), requires supplying the proper ratio of Sodium Hydroxide (NaOH) and Carbon Monoxide (CO) to Hydrogen production plant 216. A closed loop control system can be implemented to optimize the Sodium Formate (HCOONa) and Methanol (CH₃OH) production and to save power when Carbon Dioxide (CO₂) emissions are low or not present. The process may include:

The closed loop system can sense the Carbon Dioxide (CO₂) concentration in a smoke-stack of the emission source 204 with an electrochemical Carbon Dioxide (CO₂) sensor. For example, when Carbon Dioxide (CO₂) contacts the sensor, it reacts with a polymer surface resulting in electrical charge. In other embodiments, any other type of Carbon Dioxide (CO₂) sensor can be used.

The closed loop control system can include a microcontroller or computer that measures the Carbon Dioxide (CO₂) concentration with a sensor, such as mentioned above, and adjusts the production of Sodium Hydroxide (NaOH) accordingly by adjusting the power applied from the power plant 202 to the chlor-alkali membrane process 212 and the solid oxide electrolysis cell within the Carbon Dioxide (CO₂) conversion plant 208. The power saved from the above process can be sourced to a power grid or sourced to water treatment plant 210 to produce a larger volume of clean water.

In embodiments, the power plant 202 can be a permanent or temporary installation built at or near (e.g., roughly 1 km from) the location of the emission source 204, scrubber 206, Carbon Dioxide (CO₂) conversion plant 208, water treatment plant 210, chlor-alkali membrane process 212, Hydrochloric Acid production plant 214, Hydrogen production plant 216, and/or Methanol production plant 218. In embodiments, the power plant 202 can be a mobile or partially mobile system that is moved to and assembled at or near the location of the emission source 204, scrubber 206, Carbon Dioxide (CO₂) conversion plant 208, water treatment plant 210, chlor-alkali membrane process 212, Hydrochloric Acid production plant 214, Hydrogen production plant 216, and/or Methanol production plant 218. More generally, the power plant 202 can be local (e.g., positioned at or near) to the industrial processes/operations it supports. For example, the power plant can be located within 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant 202 is configured to supply a portion of electricity to a power grid.

FIG. 3 is a block diagram of a Carbon Dioxide (CO₂) conversion plant 300, in accordance with embodiments of the present disclosure. In embodiments, the Carbon Dioxide (CO₂) conversion plant 300 may include one or more solid oxide electrolysis cells (SOEC) 302. An SOEC 302 of the present invention may be configured to perform the electrochemical Carbon Dioxide (CO₂) reduction for Carbon Monoxide (CO) and Oxygen (O₂), as described, for example, in Kungas, J. Electrochem. Soc. 167, 2020 and WO2014/154253. In embodiments, the electrolyte of the SOEC 302 may include a solid ceramic material, for example stabilized zirconias, such as yttria-stabilized zirconia (YSZ, a solid solution of Y₂O₃ and ZrO₂) and scandia-stabilized zirconia (ScSZ), as well as doped cerias, such as gadolinia-doped ceria (abbreviated either as GDC) or samaria-doped ceria (SDC).

In embodiments, Carbon Dioxide (CO₂) is fed to the fuel side 304 of the SOEC 302 stack with an applied current. The fuel side 304 of the SOEC 302 may also include a cathode. Oxygen from the reaction is transported to the oxygen side 306 of the stack. The oxygen side 306 of the SOEC 302 may also include an anode. The equation that governs the Carbon Dioxide (CO₂) reduction for Carbon Monoxide (CO) and Oxygen (O₂) production is shown in Equation 1:

$${CO}_{2} \to CO + 1 / 2 {O}_{2} \qquad ( 1 )$$

In embodiments, Carbon Dioxide (CO₂) may be used to flush the oxygen side 306. Optionally, air or nitrogen may be used to flush the oxygen side 306 of the SOEC 302 stack. In preferred embodiments, Carbon Dioxide (CO₂) is used to flush the oxygen side 306, instead of air, to mitigate the leakage of undesired gases, such as Nitrogen (N₂), into the fuel side 304 of the SOEC 302. Flushing the oxygen side 306 of the SOEC 302 with Carbon Dioxide (CO₂) gas has two advantages, more specifically (1) enhancing the oxygen production concentration and (2) providing means for feeding energy into the SOEC 302. In embodiments, the SOEC 302 is operated at elevated temperatures (e.g., ˜600° C.). Inlet gas to the fuel side 304 and/or flush gas to the oxygen side 306 may be heated, for example in one or more auxiliary heaters, prior to entering the SOEC 302. In embodiments, Joule heat, i.e., the heat produced when current is passed through the SOEC 302, may supply some or all of the heat necessary for the SOEC 302. In embodiments, Joule heat, auxiliary heaters, and/or means of heating known in the art may be used in combination to provide optimum operating conditions for the SOEC 302. In embodiments, the Carbon Dioxide (CO₂) conversion plant 208 may receive power and/or thermal energy from the power plant 202.

The product stream from the fuel side 304 of the SOEC 302, for example containing Carbon Monoxide (CO) mixed with Carbon Dioxide (CO₂), may then be passed through a separation process 308 to separate the Carbon Monoxide (CO) and the Carbon Dioxide (CO₂). In embodiments, the separation process 308 may include one or more separation units, such as pressure swing adsorption (PSA), temperature swing adsorption (TSA), membrane separation, and cryogenic separation technology. Carbon Dioxide (CO₂) from the separation process 308 may be recycled within the Carbon Dioxide (CO₂) conversion plant 300, for example to the SOEC 302, and/or used in downstream processes, such as Methanol (CH₃OH) production. Carbon Monoxide (CO) from the separation process 308 may be recycled within the Carbon Dioxide (CO₂) conversion plant 300, for example to the SOEC 302, and/or used in downstream processes. such as Hydrogen (H₂) production and Methanol (CH₃OH) production.

The product stream from the oxygen side 306 of the SOEC 302, for example containing Oxygen (O₂) mixed with Carbon Dioxide (CO₂), may then be passed through a separation process 310 to separate the Oxygen (O₂) and the Carbon Dioxide (CO₂). In embodiments, the separation process 310 may include one or more separation units, such as PSA, adsorption, and membrane separation. Carbon Dioxide (CO₂) from the separation process 308 may be recycled within the Carbon Dioxide (CO₂) conversion plant 300, for example to the SOEC 302, and/or used in downstream processes, such as Methanol (CH₃OH) production. Oxygen (O₂) from the separation process 310 may be used in downstream processes, stored, further purified and/or sold.

FIG. 4 is a block diagram of a chlor-alkali membrane process 400, in accordance with embodiments of the present disclosure. The chlor-alkali membrane process 400 may use a membrane cell 404 to partition a brine solution in a first chamber 406 from a water (H₂O) solution in a second chamber 408. In embodiments, saline water is fed to a water treatment plant 402. The water treatment plant 402 may include the water treatment plant 210. In embodiments, the water treatment plant 402 produces clean water and brine. In the illustrated example, saline water, such as sea water with a salt concentration of 3.5% Sodium Chloride (NaCl), is fed to the water treatment plant 402. The water treatment plant 402 converts the saline water into clean water (e.g., <0.05% Sodium Chloride (NaCl) concentration) and brine (e.g., 7.5% Sodium Chloride (NaCl) concentration).

In embodiments, brine from the water treatment plant 402 is fed to the first chamber 406 of the chlor-alkali membrane cell 404. In embodiments, a portion of the clean water from the water treatment plant 402 may be fed to the second chamber 408 of the chlor-alkali membrane cell 404. A typical chlor-alkali membrane process can process brine concentration up to 26% Sodium Chloride (NaCl). In embodiments, brine from the water treatment plant 402 may include a Sodium Chloride (NaCl) concentration up to 26%. In embodiments, the chlor-alkali membrane process 400 may include one or more chlor-alkali membrane cells 404, for example configured to operate in series, in parallel, or a combination, therefore.

The chlor-alkali membrane cell 404 may be an ion-selective membrane configured to allow Sodium ions (Na⁺) to flow freely across the membrane between the first chamber 406 and the second chamber 408, while Chloride ions (Cl⁻) and Hydroxide ions (OH⁻) are prevented from migrating across the chlor-alkali membrane cell 404. An anode is in the first chamber 406, and a cathode is in the second chamber 408. At the anode, Chloride ions (Cl⁻) from the brine solution are oxidized to form Chlorine (Cl₂) gas. At the cathode, water (H₂O) is reduced to Hydroxide ions (OH⁻) and Hydrogen (H₂) gas, releasing Hydroxide ions (OH⁻) into the solution.

Sodium ions (Na⁺) from the brine solution in the first chamber 406 flow across the membrane toward the cathode in the second chamber 408 and combine with Hydroxide ions (OH⁻) to produce a Sodium Hydroxide (NaOH) solution. The Sodium Hydroxide (NaOH) solution may be removed as a product from the second chamber 408. The equation for the overall reaction for the electrolysis of brine is shown in Equation 2:

$$2 NaCl + 2 {H}_{2} O \to {Cl}_{2} + {H}_{2} + 2 NaOH \qquad ( 2 )$$

In embodiments, the chlor-alkali process 400 can reduce the Sodium Chloride (NaCl) concentration of brine entering the first chamber 406. The outlet stream from the first chamber 406 may, for example be reduced to a benign sea water concentrations of Sodium Chloride (NaCl) (e.g., 3.5%). Output from the first chamber 406 may be further processed in a downstream chlor-alkali membrane cell 404, fed back to the water treatment plant 402 process, or safely released back into the environment, such as into an ocean, sea, or lake comprising the same or higher concentration of Sodium Chloride (NaCl) as the output stream.

In embodiments, the Chlorine (Cl₂) gas and Hydrogen (H₂) gas generated in the chlor-alkaline membrane process 400 may be removed as a product to be stored, sold, or used in further resource production, such as in a Hydrochloric Acid production plant 410. For example, Chlorine (Cl₂) gas and Hydrogen (H₂) gas can be used to produce hydrogen chloride (HCl) gas for the conversion into Hydrochloric Acid (HCl). The reaction equation is shown in Equation 3:

$${H}_{2} + {Cl}_{2} \to 2 HCl \qquad ( 3 )$$

The chlor-alkali membrane process 400 may be energy intensive, on the order of 40 times the energy required in a Reverse Osmosis (RO) desalination process. In embodiments, the water treatment plant 402, the chlor-alkali membrane cell 404, and the Hydrochloric acid production plant 410 are configured to receive power from the power plant 202.

FIG. 5 is a block diagram of a Hydrogen production process 500 in accordance with embodiments of the present disclosure. Carbon Monoxide (CO) can be made to react with solid Sodium Hydroxide (NaOH) under pressure to produce Sodium Formate (HCOONa) and, through the thermal decomposition of Sodium Formate (HCOONa), Hydrogen (H₂) gas. For example, see U.S. Pat. No. 2,281,715, Kumar, et al., Catalysts ISSN 2073-4344 Nov. 27, 2012. In embodiments, Sodium Hydroxide (NaOH) solution, for example from the chlor-alkali membrane process 400, may be converted to a solid form through a drying process, such as a Thermal Vacuum Dehydration Chamber 502. Solid Sodium Hydroxide (NaOH) may then be fed to a Hydrogen production plant 504 here it is introduced to Carbon Monoxide (CO) in a reaction chamber 506. Sodium Formate (HCOONa) is produced by the reaction shown in Equation 4:

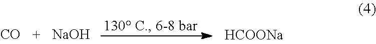

In reaction chamber 506, Carbon Monoxide (CO) is absorbed by Sodium Hydroxide (NaOH). Operating conditions for reaction chamber 506 may be optimized for yield and efficiency using techniques known in the art. For example, the reaction chamber 506 may be operated at a temperature of 130° C. and a pressure between 6-8 bar.

In embodiments, Sodium Formate (HCOONa) may be fed to a thermal decomposition chamber 508. Sodium Formate (HCOONa) slowly decomposes by hydrogen loss to produce Sodium Oxalate (Na₂C₂O₄) at 330° C., following the reaction shown in Equation 5:

$$2 HCOONa \to {( COO )}_{2} {Na}_{2} + {H}_{2} \qquad ( 5 )$$

The rate of the decomposition reaction increases markedly at 400° C., associated with a large exothermic change. Sodium Oxalate (Na₂C₂O₄) subsequently decomposes to Sodium Carbonate (Na₂CO₃) and Carbon monoxide (CO) on heating above 440° C. Both the heating rate and maximum temperature have a significant effect on the yield of Sodium Oxalate (Na₂C₂O₄). In order to maximize the yield of Sodium Oxalate (Na₂C₂O₄), Sodium Formate (HCOONa) should be heated as rapidly as possible in order to shorten the reaction time, with the optimum temperature of thermal decomposition being from 400° C. to 420° C. Overheating may cause rapid decomposition of the Sodium Oxalate (Na₂C₂O₄) to produce Sodium Carbonate (Na₂CO₃) and Carbon Monoxide (CO) gas. Hydrogen (H₂) generated in the Hydrogen production plant 504 may be sent to downstream processes for use in resource production, may be stored and/or sold. Sodium Oxalate (Na₂C₂O₄), as well as any intermediate or byproducts, such as Sodium Formate (HCOONa), Sodium Carbonate (Na₂CO₃), and Carbon monoxide (CO) generated in the Hydrogen production plant 504 may be sent to downstream processes for use in resource production, and may be stored and/or sold. In embodiments, the Hydrogen production process 500 is configured to receive power from the power plant 202.

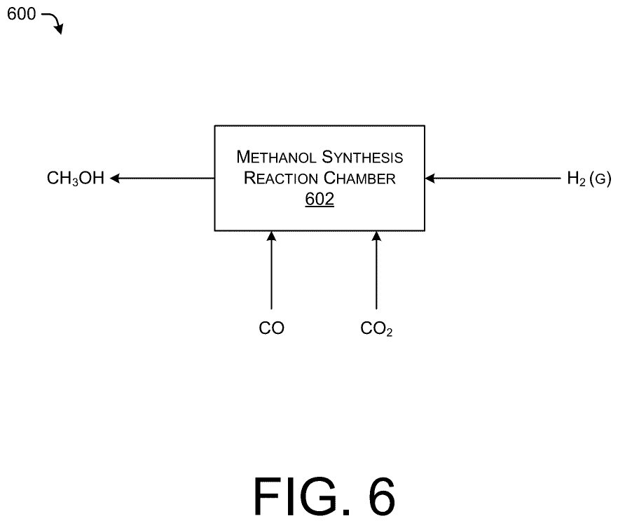

FIG. 6 is a block diagram of a Methanol production process 600, in accordance with embodiments of the present disclosure. Methanol (CH₃OH) can be produced by directly hydrogenating pure Carbon Dioxide (CO₂) with Hydrogen (H₂) with high selectivity on conventional Copper (Cu) and zinc oxide (ZnO) based catalysts (Cu/ZnO). The reaction rates are much lower than with syngas feeds because of thermodynamic limitations. This synthesis from pure Carbon Dioxide (CO₂) is also complicated because of increased water formation. In the absence of Carbon Monoxide (CO), water is produced both as the by-product of Carbon Dioxide (CO₂) hydrogenation. The increased formation of water leads to kinetic inhibition and acerated deactivation of the Cu/ZnO catalysts. Therefore, the solution is to introduce Carbon Monoxide (CO) into the reaction chamber to remove water via the Water-Gas-Shift (WGS) reaction so that the production of Methanol (CH₃OH) can be continued. The reactions for the production of Methanol (CH₃OH) are shown in Equations 6-8:

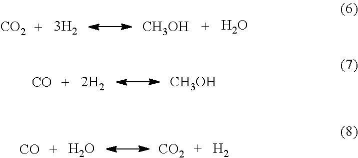

The reactions will take place over Cu/ZnO catalyst at a temperature between 200-300° C. and at a pressure between 50-100 bar. Equation 8 is the Water-Shift-Reaction (WSR). Even though Methanol (CH₃OH) can be produced by directly hydrogenating pure Carbon Dioxide (CO₂) with Hydrogen (H₂) via Equations 6 & 7 with conventional Cu/ZnO-based catalysts, the reaction rates will be terminated shortly after initiation due to the continual formation of water that leads to kinetic inhibition and the accelerated deactivation of the Cu/ZnO catalysts. The presence of Carbon Monoxide (CO) in the reaction is extremely important to continue the WSR in order to maintain the synthesis at low temperature and high pressure without the deactivation of the catalyst.

In embodiments, Carbon Dioxide (CO₂) and Carbon Monoxide (CO) and Hydrogen (H₂) are fed into a Methanol Synthesis Reaction Chamber 602. Carbon Dioxide (CO₂) and Carbon Monoxide (CO) may, for example, be fed from the Carbon Dioxide (CO₂) conversion plant 300. Hydrogen (H₂), for example, may be fed from the Hydrogen production process 500. Methanol (CH₃OH) may be stored or sold. In embodiments, the Methanol production process 600 is configured to receive power from the power plant 202.

FIG. 7 is a schematic view of a nuclear power plant system 750 (“power plant system 750”) including multiple nuclear reactors 700 (individually identified as first through twelfth nuclear reactors 700a-1, respectively) in accordance with embodiments of the present technology. The power plant system 750 can be a permanent or temporary installation built at or near (e.g., roughly 1 km from) the location of an industrial process facility or can be a mobile or partially mobile system that is moved to and assembled at or near the location of the industrial process facility. More generally, the power plant can be local (e.g., positioned at or near) to the industrial processes/operations it supports. For example, the power plant can be located within 0.4 km (0.25 mile), within 0.8 km (0.5 mile), within 3.22 km (2 miles), within 4.82 km (3 miles), or within 8.1 km (5 miles) of the industrial processes/operations it supports. In embodiments, the power plant system 750 is configured to supply a portion of electricity to a power grid.

Each of the nuclear reactors 700 can be similar to, or identical to, the nuclear reactor system 800 and/or the nuclear reactor system 900 described in detail below with reference to FIG. 8 and FIG. 9. The power plant system 750 can be “modular” in that each of the nuclear reactors 700 can be operated separately to provide an output, such as electricity or steam. In embodiments, the power plant system may include Small Modular Reactors (SMRs). The power plant system 750 can include fewer than twelve of the nuclear reactors 700 (e.g., two, three, four, five, six, seven, eight, nine, ten, or eleven of the nuclear reactors 700), or more than twelve of the nuclear reactors 700. The power plant system 750 can be a permanent installation or can be mobile (e.g., mounted on a truck, tractor, mobile platform, and/or the like). In the illustrated embodiment, each of the nuclear reactors 700 can be positioned within a common housing 751, such as a reactor plant building, and controlled and/or monitored via a control room 752.

Each of the nuclear reactors 700 can be coupled to a corresponding electrical power conversion system 740 (individually identified as first through twelfth electrical power conversion systems 740a-1, respectively). The electrical power conversion systems 740 can include one or more devices that generate electrical power or some other form of usable power from steam generated by the nuclear reactors 700. For example, the electrical power conversion systems 740 can include features that are similar or identical to the power conversion system 840 described in detail below with reference to FIG. 8. In some embodiments, multiple ones of the nuclear reactors 700 can be coupled to the same one of the electrical power conversion systems 740 and/or one or more of the nuclear reactors 700 can be coupled to multiple ones of the electrical power conversion systems 740 such that there is not a one-to-one correspondence between the nuclear reactors 700 and the electrical power conversion systems 740.

The electrical power conversion systems 740 can be further coupled to an electrical power transmission system 754 via, for example, an electrical power bus 753. The electrical power transmission system 754 and/or the electrical power bus 753 can include one or more transmission lines, transformers, and/or the like for regulating the current, voltage, and/or other characteristic(s) of the electricity generated by the electrical power conversion systems 740. The electrical power transmission system 754 can route electricity via a plurality of electrical output paths 755 (individually identified as electrical output paths 755a-n) to one or more end users and/or end uses, such as different electrical loads of an integrated energy system as described in greater detail herein.

The power plant system 750 can be configured in a first operating state to provide electricity to the water treatment plant 210 (e.g., via one or more of the electrical output paths 755 from the electrical power transmission system 754). The water treatment plant 210 can route the produced high-quality water to the power plant system 750, and the power plant system 750 can use the water to produce high-quality steam. For example, the produced water can be used as a secondary coolant in a steam generator of one or more of the nuclear reactors 700. In some embodiments, the water treatment plant 210 can be omitted and the power plant system 750 can utilize water from other sources to generate steam.

Each of the nuclear reactors 700 can further be coupled to a steam transmission system 756 via, for example, a steam bus 757. The steam bus 757 can route steam generated from the nuclear reactors 700 to the steam transmission system 756 which in turn can route the steam via a plurality of steam output paths 758 (individually identified as steam output paths 758a-n) to one or more end users and/or end uses, such as different steam inputs of an integrated energy system as described in greater detail below.

In some embodiments, the nuclear reactors 700 can be individually controlled (e.g., via the control room 752) to provide steam to the steam transmission system 756 and/or steam to the corresponding one of the electrical power conversion systems 740 to provide electricity to the electrical power transmission system 754. In some embodiments, the nuclear reactors 700 are configured to provide steam either to the steam bus 757 or to the corresponding one of the electrical power conversion systems 740, and can be rapidly and efficiently switched between providing steam to either. Accordingly, in some aspects of the present technology the nuclear reactors 700 can be modularly and flexibly controlled such that the power plant system 750 can provide differing levels/amounts of electricity via the electrical power transmission system 754 and/or steam via the steam transmission system 756. For example, where the power plant system 750 is used to provide electricity and steam to one or more industrial process—such as various components of the integrated energy systems described in the detail below—the nuclear reactors 700 can be controlled to meet the differing electricity and steam requirements of the industrial processes.

As one example, during a first operational state of an integrated energy system employing the power plant system 750, a first subset of the nuclear reactors 700 (e.g., the first through sixth nuclear reactors 700a-f) can be configured to provide steam to the steam transmission system 756 for use in the first operational state of the integrated energy system, while a second subset of the nuclear reactors 700 (e.g., the seventh through twelfth nuclear reactors 700g-1) can be configured to provide steam to the corresponding ones of the electrical power conversion systems 740 (e.g., the seventh through twelfth electrical power conversion systems 740g-1) to generate electricity for the first operational state of the integrated energy system. Then, during a second operational state of the integrated energy system when a different (e.g., greater or lesser) amount of steam and/or electricity is required, some or all the first subset of the nuclear reactors 700 can be switched to provide steam to the corresponding ones of the electrical power conversion systems 740 (e.g., the seventh through twelfth electrical power conversion systems 740g-1) and/or some or all of the second subset of the nuclear reactors 700 can be switched to provide steam to the steam transmission system 756 to vary the amount of steam and electricity produced to match the requirements/demands of the second operational state. Other variations of steam and electricity generation are possible based on the needs of the integrated energy system. That is, the nuclear reactors 700 can be dynamically/flexibly controlled during other operational states of an integrated energy system to meet the steam and electricity requirements of the operational state.

In contrast, some conventional nuclear power plant systems can typically generate a fixed amount of either steam or electricity for output, and cannot be modularly controlled to provide varying levels of steam and electricity for output. Moreover, it is typically difficult (e.g., expensive, time consuming, etc.) to switch between steam generation and electricity generation in conventional nuclear power plant systems. Specifically, for example, it is typically extremely time consuming to switch between steam generation and electricity generation in prototypical large nuclear power plant systems.

The nuclear reactors 700 can be individually controlled via one or more operators and/or via a computer system. Accordingly, many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described herein. The technology can be embodied in a special-purpose computer, controller or data processor that is specifically programmed, configured or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, minicomputers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described herein may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

FIG. 8 and FIG. 9 illustrate representative nuclear reactors that may be included in embodiments of the present technology. FIG. 8 is a partially schematic, partially cross-sectional view of a nuclear reactor system 800 configured in accordance with embodiments of the present technology. The system 800 can include a power module 802 having a reactor core 804 in which a controlled nuclear reaction takes place. Accordingly, the reactor core 804 can include one or more fuel assemblies 801. The fuel assemblies 801 can include fissile and/or other suitable materials. Heat from the reaction generates steam at a steam generator 830, which directs the steam to a power conversion system 840. The power conversion system 840 generates electrical power, and/or provides other useful outputs, such as super-heated steam. A sensor system 850 is used to monitor the operation of the power module 802 and/or other system components. The data obtained from the sensor system 850 can be used in real time to control the power module 802, and/or can be used to update the design of the power module 802 and/or other system components.

The power module 802 includes a containment vessel 810 (e.g., a radiation shield vessel, or a radiation shield container) that houses/encloses a reactor vessel 820 (e.g., a reactor pressure vessel, or a reactor pressure container), which in turn houses the reactor core 804. The containment vessel 810 can be housed in a power module bay 856. The power module bay 856 can contain a cooling pool 803 filled with water and/or another suitable cooling liquid. The bulk of the power module 802 can be positioned below a surface 805 of the cooling pool 803. Accordingly, the cooling pool 803 can operate as a thermal sink, for example, in the event of a system malfunction.

A volume between the reactor vessel 820 and the containment vessel 810 can be partially or completely evacuated to reduce heat transfer from the reactor vessel 820 to the surrounding environment (e.g., to the cooling pool 803). However, in other embodiments the volume between the reactor vessel 820 and the containment vessel 810 can be at least partially filled with a gas and/or a liquid that increases heat transfer between the reactor vessel 820 and the containment vessel 810. For example, the volume between the reactor vessel 820 and the containment vessel 810 can be at least partially filled (e.g., flooded with the primary coolant 807) during an emergency operation.

Within the reactor vessel 820, a primary coolant 807 conveys heat from the reactor core 804 to the steam generator 830. For example, as illustrated by arrows located within the reactor vessel 820, the primary coolant 807 is heated at the reactor core 804 toward the bottom of the reactor vessel 820. The heated primary coolant 807 (e.g., water with or without additives) rises from the reactor core 804 through a core shroud 806 and to a riser tube 808. The hot, buoyant primary coolant 807 continues to rise through the riser tube 808, then exits the riser tube 808 and passes downwardly through the steam generator 830. The steam generator 830 includes a multitude of conduits 832 that are arranged circumferentially around the riser tube 808, for example, in a helical pattern, as is shown schematically in FIG. 8. The descending primary coolant 807 transfers heat to a secondary coolant (e.g., water) within the conduits 832, and descends to the bottom of the reactor vessel 820 where the cycle begins again. The cycle can be driven by the changes in the buoyancy of the primary coolant 807, thus reducing or eliminating the need for pumps to move the primary coolant 807.

The steam generator 830 can include a feedwater header 831 at which the incoming secondary coolant enters the steam generator conduits 832. The secondary coolant rises through the conduits 832, converts to vapor (e.g., steam), and is collected at a steam header 833. The steam exits the steam header 833 and is directed to the power conversion system 840.

The power conversion system 840 can include one or more steam valves 842 that regulate the passage of high pressure, high temperature steam from the steam generator 830 to a steam turbine 843. The steam turbine 843 converts the thermal energy of the steam to electricity via a generator 844. The low-pressure steam exiting the turbine 843 is condensed at a condenser 845, and then directed (e.g., via a pump 846) to one or more feedwater valves 841. The feedwater valves 841 control the rate at which the feedwater re-enters the steam generator 830 via the feedwater header 831. In other embodiments, the steam from the steam generator 830 can be routed for direct use in an industrial process, such as a hydrogen and oxygen production plant, a chemical production plant, and/or the like, as described in detail in this application. Accordingly, steam exiting the steam generator 830 can bypass the power conversion system 840.

The power module 802 includes multiple control systems and associated sensors. For example, the power module 802 can include a hollow cylindrical reflector 809 that directs neutrons back into the reactor core 804 to further the nuclear reaction taking place therein. Control rods 813 are used to modulate the nuclear reaction, and are driven via fuel rod drivers 815. The pressure within the reactor vessel 820 can be controlled via a pressurizer plate 817 (which can also serve to direct the primary coolant 807 downwardly through the steam generator 830) by controlling the pressure in a pressurizing volume 819 positioned above the pressurizer plate 817.

The sensor system 850 can include one or more sensors 851 positioned at a variety of locations within the power module 802 and/or elsewhere, for example, to identify operating parameter values and/or changes in parameter values. The data collected by the sensor system 850 can then be used to control the operation of the system 800, and/or to generate design changes for the system 800. For sensors positioned within the containment vessel 810, a sensor link 852 directs data from the sensors to a flange 853 (at which the sensor link 852 exits the containment vessel 810) and directs data to a sensor junction box 854. From there, the sensor data can be routed to one or more controllers and/or other data systems via a data bus 855.

FIG. 9 is a partially schematic, partially cross-sectional view of a nuclear reactor system 900 (“system 900”) configured in accordance with additional embodiments of the present technology. In some embodiments, the system 900 can include some features that are at least generally similar in structure and function, or identical in structure and function, to the corresponding features of the system 800 described in detail above with reference to FIG. 8, and can operate in a generally similar or identical manner to the system 800.

In the illustrated embodiment, the system 900 includes a reactor vessel 920 and a containment vessel 910 surrounding/enclosing the reactor vessel 920. In some embodiments, the reactor vessel 920 and the containment vessel 910 can be roughly cylinder-shaped or capsule-shaped. The system 900 further includes a plurality of heat pipe layers 911 within the reactor vessel 920. In the illustrated embodiment, the heat pipe layers 911 are spaced apart from and stacked over one another. In some embodiments, the heat pipe layers 911 can be mounted/secured to a common frame 912, a portion of the reactor vessel 920 (e.g., a wall thereof), and/or other suitable structures within the reactor vessel 920. In other embodiments, the heat pipe layers 911 can be directly stacked on top of one another such that each of the heat pipe layers 911 supports and/or is supported by one or more of the other ones of the heat pipe layers 911.

In the illustrated embodiment, the system 900 further includes a shield or reflector region 914 at least partially surrounding a core region 916. The heat pipes layers 911 can be circular, rectilinear, polygonal, and/or can have other shapes, such that the core region 916 has a corresponding three-dimensional shape (e.g., cylindrical, spherical). In some embodiments, the core region 916 is separated from the reflector region 914 by a core barrier 915, such as a metal wall. The core region 916 can include one or more fuel sources, such as fissile material, for heating the heat pipes layers 911. The reflector region 914 can include one or more materials configured to contain/reflect products generated by burning the fuel in the core region 916 during operation of the system 900. For example, the reflector region 914 can include a liquid or solid material configured to reflect neutrons and/or other fission products radially inward toward the core region 916. In some embodiments, the reflector region 914 can entirely surround the core region 916. In other embodiments, the reflector region 914 may partially surround the core region 916. In some embodiments, the core region 916 can include a control material 917, such as a moderator and/or coolant. The control material 917 can at least partially surround the heat pipe layers 911 in the core region 916 and can transfer heat therebetween.

In the illustrated embodiment, the system 900 further includes at least one heat exchanger 930 (e.g., a steam generator) positioned around the heat pipe layers 911. The heat pipe layers 911 can extend from the core region 916 and at least partially into the reflector region 914, and are thermally coupled to the heat exchanger 930. In some embodiments, the heat exchanger 930 can be positioned outside of or partially within the reflector region 914. The heat pipe layers 911 provide a heat transfer path from the core region 916 to the heat exchanger 930. For example, the heat pipe layers 911 can each include an array of heat pipes that provide a heat transfer path from the core region 916 to the heat exchanger 930. When the system 900 operates, the fuel in the core region 916 can heat and vaporize a fluid within the heat pipes in the heat pipe layers 911, and the fluid can carry the heat to the heat exchanger 930. The heat pipes in the heat pipe layers 911 can then return the fluid toward the core region 916 via wicking, gravity, and/or other means to be heated and vaporized once again.

In some embodiments, the heat exchanger 930 can be similar to the steam generator 830 of FIG. 8 and, for example, can include one or more helically-coiled tubes that wrap around the heat pipe layers 911. The tubes of the heat exchanger 930 can include or carry a working fluid (e.g., a coolant such as water or another fluid) that carries the heat from the heat pipe layers 911 out of the reactor vessel 920 and the containment vessel 910 for use in generating electricity, steam, and/or the like. For example, in the illustrated embodiment the heat exchanger 930 is operably coupled to a turbine 943, a generator 944, a condenser 945, and a pump 946. As the working fluid within the heat exchanger 930 increases in temperature, the working fluid may begin to boil and vaporize. The working fluid (e.g., steam) may be used to drive the turbine 943 to convert the thermal potential energy of the working fluid into electrical energy via the generator 944. The condenser 945 can condense the working fluid after it passes through the turbine 943, and the pump 946 can direct the working fluid back to the heat exchanger 930 where it can begin another thermal cycle. In other embodiments, steam from the heat exchanger 930 can be routed for direct use in an industrial process, such as a resource production plant, described in detail above. Accordingly, steam exiting the heat exchanger 930 can bypass the turbine 943, the generator 944, the condenser 945, the pump 946, etc.

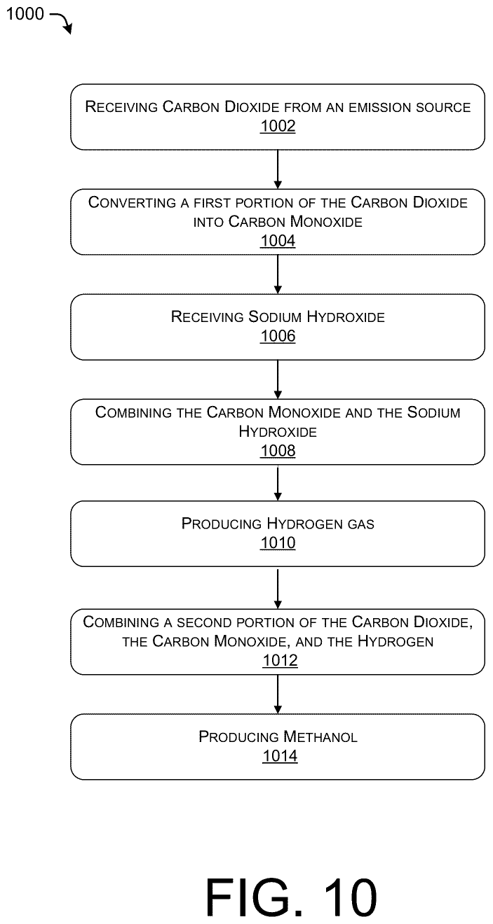

FIG. 10 illustrates and example process 1000 for producing Methanol. In various examples, the process 1000 can be performed by an Integrated Energy System (IES), such as the IES 100 and 200. The IES may include a power plant, such as the power plant 102, 202, and the power plant system 750. In embodiments, the power plant comprises a nuclear power module. The IES may also include a resource production plant, such as the resource production plant 108. In embodiments, the resource production plant may include the one or more chemical production plants or sub-plants. The resource production plant may include: an emission source, such as emission source 204, a scrubber, such as scrubber 206, a Carbon Dioxide (CO₂) conversion plant, such as Carbon Dioxide (CO₂) conversion plant 208, a water treatment plant, such as water treatment plant 210, a chlor-alkali membrane process, such as chlor-alkali membrane process 212, a Hydrochloric Acid production plant, such as Hydrochloric Acid production plant 214, a Hydrogen production plant, such as Hydrogen production plant 216, and a Methanol production plant, such as Methanol production plant 218. In embodiments, the resource production plants and/or chemical production plants and sub-plants are of the process 1000 configured to receive electricity from the power plant. In embodiments, the power plant is configured to provide power to the water treatment plant, an electrolysis cell, and a reaction chamber. In embodiments, the nuclear power module is local to the emission source, the water treatment plant, the electrolysis cell, the resource production plant, or the reaction chamber.

At 1002 Carbon Dioxide (CO₂) is received from an emission source, such as a natural-gas or coal-fired power generation plant, a chemical product production plants, an oil refinery, an ore processing plant, or a steel processing plant.

At 1004 a first portion of the Carbon Dioxide (CO₂) is converted into Carbon Monoxide (CO). For example, the Carbon Dioxide (CO₂) is converted into the Carbon Monoxide (CO) in a Solid Oxide Electrolysis Cell (SOEC). In embodiments, the SOEC is further configured to convert the Carbon Dioxide (CO₂) into Carbon Monoxide (CO) and Oxygen (O₂).

At 1006 Sodium Hydroxide (NaOH) is received to the process 1000. In embodiments, the Sodium Hydroxide (NaOH) is received from a chlor-alkali membrane cell. In embodiments, the chlor-alkali membrane cell converts brine, a Sodium Chloride (NaCl) solution, from a water treatment plant into the Sodium Hydroxide (NaOH). In embodiments, the water treatment plant includes Reverse Osmosis to produce clean water and brine. Brine from the water treatment plant may be fed to the chlor-alkali membrane cell to produce Sodium Hydroxide.

At 1008 the Carbon Monoxide (CO) and the Sodium Hydroxide (NaOH) are combined. In embodiments, the Carbon Monoxide (CO) and the Sodium Hydroxide (NaOH) react to form Sodium Formate (HCOONa).

At 1010, Hydrogen (H₂) gas is produced by the thermal decomposition of Sodium Formate. The resulting products of the thermal decomposition of Sodium Formate (HCOONa) include Sodium Oxalate (Na₂C₂O₄).

At 1012, and using a reaction chamber, a second portion of the Carbon Dioxide (CO₂), the Carbon Monoxide (CO), and the Hydrogen (H₂) are combined.

At 1014 Methanol is produced from the reaction of the Carbon Dioxide (CO₂), the Carbon Monoxide (CO), and the Hydrogen (H₂).

### CONCLUSION

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims.

What is claimed is:

1. An Integrated Energy System (IES) comprising:
a power plant,
an emission source providing Carbon Dioxide,
a water treatment plant configured to produce Sodium Hydroxide from salt water,
an electrolysis cell configured to convert a first portion of the Carbon Dioxide into Carbon Monoxide,
a resource production plant configured to combine the Carbon Monoxide with Sodium Hydroxide to produce Hydrogen gas, and
a reaction chamber configured to receive a second portion of the Carbon Dioxide, the Carbon Monoxide, and the Hydrogen to produce Methanol.

2. The IES of claim 1 wherein the power plant is configured to provide power to the water treatment plant, the electrolysis cell, the resource production plant, and the reaction chamber.

3. The IES of claim 1 wherein the power plant comprises a nuclear power module.

4. The IES of claim 3 wherein the nuclear power module is local to the emission source, the water treatment plant, the electrolysis cell, the resource production plant, or the reaction chamber.

5. The IES of claim 1 wherein the emission source comprises a natural-gas or coal-fired power generation plant, a chemical product production plants, an oil refinery, an ore processing plant, or a steel processing plant.

6. The IES of claim 1 wherein the salt water undergoes Reverse Osmosis in the water treatment plant to produce clean water and brine.

7. The IES of claim 6 wherein the brine is fed to a chlor-alkali membrane cell to produce the Sodium Hydroxide.

8. The IES of claim 1 wherein the electrolysis cell is further configured to convert the Carbon Dioxide into Oxygen.

9. The IES of claim 1 wherein the resource production plant is further configured to produce Sodium Oxalate.

10. A Chemical Processing Plant, comprising a one or more sub-plants configured to:
receive Carbon Dioxide from an emission source,
convert a first portion of the Carbon Dioxide into Carbon Monoxide,
receive Sodium Hydroxide,
combine the Carbon Monoxide and the Sodium Hydroxide to produce Hydrogen gas, and
combine, using a reaction chamber, a second portion of the Carbon Dioxide, the Carbon Monoxide, and the Hydrogen to produce Methanol.

11. The Chemical Processing Plant of claim 10 wherein the emission source comprises a natural-gas or coal-fired power generation plant, a chemical product production plants, an oil refinery, an ore processing plant, or a steel processing plant.

12. The Chemical Processing Plant of claim 10 wherein the Carbon Dioxide is converted into the Carbon Monoxide in a Solid Oxide Electrolysis Cell.

13. The Chemical Processing Plant of claim 10 wherein the Sodium Hydroxide is received from a chlor-alkali membrane cell.

14. The Chemical Processing Plant of claim 13 wherein the chlor-alkali membrane cell converts brine into the Sodium Hydroxide.

15. The Chemical Processing Plant of claim 14 wherein the brine is produced in a desalination plant.

16. The Chemical Processing Plant of claim 15 wherein the desalination plant comprises Reverse Osmosis.

17. A method for producing Methanol comprising:
receiving Carbon Dioxide from an emission source,
converting a first portion of the Carbon Dioxide into Carbon Monoxide,
receiving Sodium Hydroxide,
combining the Carbon Monoxide and the Sodium Hydroxide to produce Hydrogen gas, and
combining, in a reaction chamber, a second portion of the Carbon Dioxide, the Carbon Monoxide, and the Hydrogen to produce Methanol.

18. The method of claim 17 wherein the Carbon Dioxide is converted into the Carbon Monoxide in a Solid Oxide Electrolysis Cell.

19. The method of claim 17 wherein the Sodium Hydroxide is received from a chlor-alkali membrane cell.

20. The method of claim 19 wherein the chlor-alkali membrane cell converts brine into the Sodium Hydroxide.
